(19) United States
(12) Patent Application Publication
(10) Pub. No.: US 2007/0082914 A1
(43) Pub. Date: Apr. 12, 2007

(54) 4-(4-Methylpiperazin-1-ylmethyl)-n-[4-methyl-3-(4-pyridin-3-yl)pyrimidin-2-ylamino)phenyl]-benzamide for treating pulmonary fibrosis

(76) Inventor: Joseph Alexander Lasky, New Orleans, LA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/518,988

(22) PCT Filed: Jun. 17, 2003
(86) PCT No.: PCT/IB03/02794

§ 371 Date: Nov. 7, 2006

Related U.S. Application Data
(60) Provisional application No. 60/392,588, filed on Jun. 28, 2002.

(51) Int. Cl. A61K 31/506

(52) U.S. Cl. 514/252.18

(57) ABSTRACT

4-(4-methylpiperazin-1-ylmethyl)-N-[4-methyl-3-(4-pyridin-3-yl)pyrimidin-2-ylamino)phenyl]-benzamide of the formula I 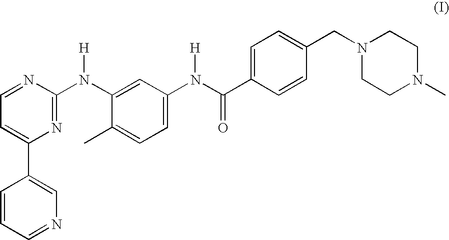 or a pharmaceutically acceptable salt thereof can be used in the treatment of pulmonary fibrosis.

The invention relates to the use of (hereinafter: “COMPOUND I”) or a pharmaceutically acceptable salt thereof for the manufacture of pharmaceutical compositions for use in the treatment of pulmonary fibrosis, to the use of COMPOUND I or a pharmaceutically acceptable salt thereof in the treatment of pulmonary fibrosis, and to a method of treating warm-blooded animals including humans suffering from pulmonary fibrosis by administering to a said animal in need of such treatment an effective dose of COMPOUND I or a pharmaceutically acceptable salt thereof.

The lungs are stimulated by a variety of antigens, mitogens, metals, chemicals, and fumes. After lung injury, acute inflammation and tissue repair mechanisms are engaged to halt the injurious stimulus, remove infectious organisms if present, and initiate immediate repair to crucial membranes that function to provide gas exchange for survival. This usually results in the eventual return of the organ to normal function. However, in chronic tissue injury, with repeat episodes of inflammation, many of the control mechanisms involved in this otherwise well orchestrated process are bypassed. Continued repair results in disorder of the tissue, distorted matrix deposition, mesenchymal cell proliferation and alteration to normal lung structure, with compromised gas exchange function; this overall process is known as pulmonary fibrosis. Pulmonary fibrosis is a common pathologic reaction to non-specific post-inflammatory local fibrosis as well as specific processes that occur in interstitial pneumonias. Fibrotic changes cause functional dysfunction and are categorized as disease entities (e.g. interstitial pneumonia and bronchiectasis).

Fibrosis of the lung may occur in five distinct patterns: bronchial, interstitial, parenchymal, pleural, and vascular. The different patterns will to a great extent determine the type of functional disability, and may often coexist.
Bronchial fibrosis will produce functional changes associated with diffuse obstructive emphysema.
Interstitial fibrosis will produce essentially diffusion disturbances.
Vascular fibrosis will produce pulmonary hypertension.
Pleural fibrosis will produce some degree of ventilatory disturbance, as will advanced degrees of parenchymal fibrosis.

The interstitial lung diseases (ILDs) represent a large number of conditions that involve the parenchyma of the lung—the alveoli, the alveolar epithelium, the capillary endothelium, and the spaces between these structures, as well as the perivascular and lymphatic tissues. This heterogeneous group of disorders is classified together because of similar clinical, roentgenographic, physiologic, or pathologic manifestations. These disorders are often associated with considerable morbidity and mortality, and there is little consensus regarding the best management of most of them.

Interstitial lung diseases have been difficult to classify because more than 200 known individual diseases are characterized by diffuse parenchymal lung involvement, either as the primary condition or as a significant part of a multi-organ process, as may occur in the connective tissue diseases (CTDs). Some of them progress into end-stage fibrotic lesions unresponsive to corticosteroid therapy. However, there are differences in the sites of fibrosis and in the distribution of fibrosis among them.

Typical examples of Interstitial lung diseases are fibrosis with pulmonary sarcoidosis, and fibrosis with chronic-type interstitial pneumonia, as seen in patients with idiopathic pulmonary fibrosis (IPF), usual interstitial pneumonia (UIP), and non-specific interstitial pneumonia (NSIP).

The classification and meaning of all the lung diseases covered by the present invention are described by Nagai et al. (Heterogeneity of pulmonary fibrosis; Curr. Opin. Pulm. Med. 2001, 7:262-71), A-L. A. Katzenstein (Idiopathic Pulmonary Fibrosis; Am. J. Respir. Crit. Care Med., 1998, 157:1301-15,) and H. Y. Reynolds (Interstitial Lung Diseases; Harrison's Principles of Internal Medicine—McGraw-Hill edition—ISBN: 0-07-020293-1; 14^{Th} edition vol. 2, Chapter 259), the contents of which are hereby incorporated by reference.

Thus, despite the variation in cause and presentation, this diverse group of diseases shares common radiographic and physiologic features.

The strong clinical similarities between patients with pulmonary fibrosis are paralleled by common pathologic features. Although there are histologic patterns that may indicate or at least suggest specific individual disease entities, such as sarcoidosis or hypersensitivity pneumonitis, certain pathologic characteristics are shared by most patients with pulmonary fibrosis. There is increased collagen deposition in the periphery of the lung, with thickening of alveolar walls. This increased collagen is associated with increased numbers of fibroblasts in the interstitium and in the alveolar space itself.

A universal feature of pulmonary fibrosis attributable to a wide variety of causes is alterations in the epithelial cells that define the alveolar space. In pulmonary fibrosis, type I alveolar epithelial cells are lost and the alveolar surface is covered by hyperplastic type II cells. Finally, a consistent finding in patients with active pulmonary fibrosis and in animal models of fibrotic lung diseases is the accumulation of increased numbers of immune and inflammatory cells in areas undergoing fibrosis. The specific pattern of leukocytes is related both to the etiology and the prognosis of the fibrotic process. Patients with Interstitial Pulmonary Fibrosis (IPF) generally demonstrate a neutrophil-predominant alveolitis. The consistent observation that recruited immune and inflammatory cells are present during active pulmonary fibrosis has provided important support for a commonly held hypothesis regarding the etiology of this process: that pulmonary fibrosis is the result of aberrant repair after an initial inflammatory insult.

Pulmonary fibrosis is a major source of morbidity and mortality. Patients typically present with symptoms of cough and dyspnea; when the condition progresses, chronic respiratory failure and cor pulmonale often ensue. Although some forms of pulmonary fibrosis of known origin may have a better prognosis, idiopathic pulmonary fibrosis (IPF) is a progressive condition that rarely, if ever, remits spontaneously. In large series, the 5-year survival of patients with IPF was less than 50%. Unfortunately, despite intensive investigation, the results of therapy for IPF have remained poor.

IPF or cryptogenic fibrosing alveolitis, is a complex pulmonary disorder and is the most common form of idiopathic interstitial pneumonia. Although much basic and clinical research has been done with the aim of understanding its pathogenesis, progression, and treatment, only a few reports in the English literature have established an association of IPF with pulmonary malignancy. Idiopathic pulmonary fibrosis is considered a pulmonary disease of unknown cause, characterized by parenchyma inflammation (alveolitis) and progressive interstitial fibrosis. IPF is slowly progressive and results in death. The estimated prevalence of IPF is wide, ranging from 3 to 29 cases per 100,000 population. This wide range is partly caused by the lack of a uniform definition of IPF and differences in clinical versus histologic criteria for its diagnosis. Most patients are in the sixth and seventh decades of life. The male-to-female ratio ranges from approximately 1:1 to 2:1.

Histologically, IPF is characterized by a heterogeneous appearance with alternating zones of normal lung, active fibrosis, and honeycomb change. Pathologic changes are pronounced in the sub-pleural regions and are accompanied by patchy inflammation. The traditional mainstay of treatment of IPF has been corticosteroid in conjunction with other immunosuppressive agents. Recently, colchicine and other anti-fibrotic agents have been used in treatment. First-line therapy with corticosteroids offers only a 15% to 20% response rate despite very significant side effects. More aggressive immunosuppressive therapy with cytotoxic agents has had only a modest impact on the outcome of the disease.

Thus, there are currently no effective therapies for the treatment of IPF.

The instant invention is a response to the need for an alternative therapy in the treatment of pulmonary fibrosis, especially interstitial fibrosis and in particular idiopathic pulmonary fibrosis.

It has now surprisingly been demonstrated that pulmonary fibrosis can be successfully treated with COMPOUND I, or pharmaceutically acceptable salt thereof.

The present invention thus concerns the use of 4-(4-methylpiperazin-1-ylmethyl)-N-[4-methyl-3-(4-pyridin-3-yl)pyrilmidin-2-ylamino)phenyl]-benzamide having the formula I 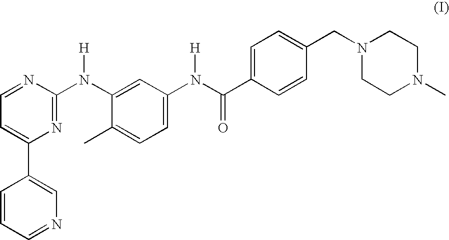
or a pharmaceutically acceptable salt thereof, for the manufacture of a medicament for treating pulmonary fibrosis.

The present invention particularly concerns the use of COMPOUND I for the manufacture of a medicament for treating interstitial fibrosis.

The present invention most particularly concerns the use of COMPOUND I for the manufacture of a medicament for treating idiopathic pulmonary fibrosis.

4-(4methylpiperazin-1-ylmethyl)-N-[4-methy-3-(4-pyridin-3-yl)pyrimidin-2-ylamino)phenyl]-benzamide or a pharmaceutically acceptable salt or β-crystal form thereof will be referred herein as COMPOUND I (also known as “Imatinib” [International Non-proprietary Name]).

The preparation of COMPOUND I and the use thereof, especially as an anti-tumour agent, are described in Example 21 of European patent application EP-A0 564 409, which was published on 6 Oct. 1993, and in equivalent applications and patents in numerous other countries, e.g. in U.S. Pat. No. 5,521,184 and in Japanese patent 2706682.

Pharmaceutically acceptable salts of COMPOUND I are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid.

The monomethanesulfonic acid addition salt of COMPOUND I (hereinafter “COMPOUND I mesylate” or “imatinib mesylate”) and a preferred crystal form thereof are described in PCT patent application WO99/03854 published on Jan. 28, 1999. Possible pharmaceutical preparations, containing an effective amount of COMPOUND I are also described in WO99/03854.

The term “treatment” as used herein means curative treatment and prophylactic treatment.

The term “curative” as used herein means efficacy in treating ongoing episodes of pulmonary fibrosis.

The term “prophylactic” means the prevention of the onset or recurrence of pulmonary fibrosis.

Depending on species, age, individual condition, mode of administration, and the clinical picture in question, effective doses, for example daily doses of about 100-1000 mg, e.g. 200 to 800 mg, preferably 200-600 mg, especially 400 mg, are administered to warm-blooded animals of about 70 kg bodyweight. For adult patients with unresectable pulmonary fibrosis, a starting dose of 400 mg daily can be recommended. For patients with an inadequate response after an assessment of response to therapy with 400 mg daily, dose escalation can be safely considered and patients may be treated as long as they benefit from treatment and in the absence of limiting toxicities.

The invention relates also to a method for administering to a human subject having pulmonary fibrosis, a COMPOUND I or a pharmaceutically acceptable salt thereof, which comprises administering a pharmaceutically effective amount of COMPOUND I or a pharmaceutically acceptable salt thereof to the human subject. Preferably administered once daily for a period exceeding 3 months. The invention relates especially to such method wherein a daily dose of 100 to 1000 mg, e.g. 200 to 800 mg, especially 400-600 mg, preferably 400 mg, of COMPOUND I mesylate is administered.

It can be shown by established test models that the COMPOUND I or a pharmaceutically acceptable salt thereof, results in a more effective prevention or preferably treatment of pulmonary fibrosis. COMPOUND I or a pharmaceutically acceptable salt thereof has significant fewer side effects as a current therapy. Furthermore, COMPOUND I or a pharmaceutically acceptable salt thereof, results in beneficial effects in different aspect of pulmonary fibrosis such as, e.g. inflammation (e.g. mural inflammation, interstitial inflammation, alveolar inflammation), fibroblast proliferation, lung collagen accumulation, alveolar wall thickening, interstitial remodeling or lung extracellular matrix deposition and remodeling, lung scarring, honeycombing.

COMPOUND I or a pharmaceutically acceptable salt thereof, shows an unexpected high potency to prevent or eliminate pulmonary fibrosis because of its unexpected multifunctional activity, and its activity on different aspects of pulmonary fibrosis.

The person skilled in the pertinent art is fully enabled to select a relevant test model to prove the hereinbefore and hereinafter indicated therapeutic indications and beneficial effects (i.e. good therapeutic margin, and other advantages mentioned herein). The pharmacological activity is, for example, demonstrated in in vitro and in vivo test procedures, or in a clinical study as essentially described hereinafter. For example, in vivo tests can show that the COMPOUND I or a pharmaceutically acceptable salt thereof, inhibits the formation of asbestos-induced lung scarring in mice or significantly reduces vanadium-induced pulmonary fibrosis (i.e. inhibition of fibroblast proliferation, reduction of the hydroxyproline accumulation) in mice (same protocol as described by Driscoll K E et al. in Toxico. I Appl. Pharmacol. (1992) 116:30-7). A number of transgenic mice have been developed that can be used for confirming the efficacy of the COMPOUND I for treating lung fibrosis (for review see Ho Y. S. (1994) Transgenic models for the study of lung biology and diseases; Am. J. Physiol. 266, L139-L353). The following Example illustrates the invention described above, but is not, however, intended to limit the scope of the invention in any way.

### Example 1

### A Phase II, Randomized, Double-Blind, Placebo-Controlled Study of the Safety and Clinical Effects of COMPOUND I (imatinib mesylate) Administered Orally to Patients with Idiopathic Pulmonary Fibrosis

The objectives of this study of patients with idiopathic pulmonary fibrosis who have failed treatment with corticosteroids are as follows:
To assess the safety of COMPOUND I (imatinib mesylate) administered orally with placebo in patients with IPF.
To assess the effect of biologic and clinical markers of lung function, including oxygenation, diffusion, lung volumes, exercise tolerance and high-resolution tomography of COMPOUND I (imatinib mesylate) administered orally compared with placebo.

Study Design: The study designs a phase II, randomized, double-blind, placebo-controlled study of the safety and clinical effects of COMPOUND I (imatinib mesylate) administered orally to patients with idiopathic pulmonary fibrosis. Study subjects with IPF are treated with COMPOUND I (imatinib mesylate) corresponding to 600 mg of COMPOUND I free base orally once per day versus a placebo control for a period of 48 weeks.

Number of patients and population: Thirty patients are enrolled into the trial in total (15 active drug and 15 placebo). The study population consists of male and female outpatients with IPF who have failed to respond to an adequate course of steroid therapy.

Inclusion criteria: In the absence of clinical features suggesting infection, neoplasm, sarcoidosis, collagen vascular disease, or exposure to known fibrogenic environmental factors, patients must fulfill all of the following study criteria to be eligible for enrollment into the study:
1) Clinical symptoms consistent with IPF with onset between 3 months and 48 months prior to screening.
2) Worsening as demonstrated by one any one of the following within the past year: >10% decrease in percent predicted FVC, worsening chest X-ray, or worsening, dyspnea at rest or on exertion.
3) Age 20 through 79, inclusive. Patients aged 20-50 must have diagnosis by either open or VATS lung biopsy to be eligible.
4) Diagnosis must be made by high resolution computed tomographic scan showing definite or probable IPF AND either of the following:
   a) Open or VATS lung biopsy showing definite or probable UIP.
   b) Non-diagnostic Tran bronchial biopsy to exclude other conditions (including granulomatous disease and malignancies) AND abnormal pulmonary function tests (reduced FVC or decreased DLCO or impaired gas exchange with rest or exercise AND 2 of the following—Age>50 years
      Insidious onset of otherwise unexplained dyspnea or exertion
      bibasilar, inspiratory crackles on examination
5) Failure to show improvement after an adequate course of steroids.
6) FVC>50% and <90% of predicted value at Baseline.
7) DLCO>30% of predicted value at Screening.
8) PaO2>60 mmHg at rest on room air at Baseline
9) Able to understand and sign a written informed consent form and comply with the requirements of the study.

Exclusion criteria: Patients with any of the following are excluded from the study:
1) History of clinically significant environmental exposure known to cause pulmonary fibrosis.
2) Diagnosis of connective tissue disease.
3) FEV1/FVC ratio<0.6 at screening (post-bronchodilator).
4) Residual volume>120% predicted at Screening.
5) Evidence of active infection
6) Any condition other than IPF, which, in the opinion of the site principal investigator, is likely to result in the death of the patient within the next year.
7) History of unstable or deteriorating cardiac or neurologic disease.
8) Pregnancy or lactation.
9) Prior treatment with Interferon gamma or beta, or with endothelin receptor blockers. 10)

Investigational therapy for any indication within 28 days prior to treatment.

Creatinine>1.5×ULN at Screening.

Hematology outside of specified limits: WBC<2,500/mm³, hematocrit<30% or >59%, platelets<1000,000/mm³ at Screening.

Any of the following liver function test criteria above specified limits: Total bilirubin>1.5×ULN, aspartate or alanine aminotransferases (AST, SGOT or ALT, SGPT)>3×ULN; alkaline phosphatase>3×ULN, and albumin<3.0 mg/dL at Screening.

Treatment regimen: The treatment regimen consist of 600 mg of COMPOUND I (imatinib mesylate) orally once per day for a period of 48 weeks. Drug level assessments would be beneficial.

Study duration/timelines: Patient accrual occurs over a period of approximately 6 months.

Criteria for Evaluation:

Efficacy is judged by the following endpoints:

Change in percent predicted FVC at 48 weeks.

Change from baseline in percent predicted DLCO at 48 weeks.

Change from baseline in the resting arterial blood gas assessment of A-a gradient at 48 weeks.

Change in the number of meters walked in a 6-minute walk test at 48 weeks.

Change form baseline in HRCT scans at 48 weeks.

Change from baseline in dyspnea (MRC, BDI/TDI, and the UCSD SOBQ) at 48 weeks.

Safety: Clinical evaluations are conducted every 4 weeks. Laboratory evaluation is conducted at study site laboratories and includes a CBC with platelet count, serum chemistry profile including liver enzyme levels, urinalysis with microscopic evaluation, and prothrombin/partial thromboplastin times.

Taken together, these results suggest that COMPOUND I has an unexpected potential for the treatment of pulmonary fibrosis.

### Example 2

### Capsules with 4[-(4-methyl-1-piperazin-1-ylmethyl)-N-[4-methyl-3-[[4-(3-pyridinyl)-2-pyrimidinyl]amino]phenyl]benzamide methanesulfonate (optionally in its β-crystal form)

Capsules containing 119.5 mg of the compound named in the title (=COMPOUND I mesylate) corresponding to 100 mg of COMPOUND I (free base) as active substance are prepared in the following composition:

| | | |
|---|---|---|
| COMPOUND I mesylate | 119.5 | mg |
| Cellulose MK GR | 92 | mg |
| Crospovidone XL | 15 | mg |
| Aerosil 200 | 2 | mg |
| Magnesium stearate | 1.5 | mg |
| | 230 | mg |

The capsules are prepared by mixing the components and filling the mixture into hard gelatin capsules, size 1.

### Example 3

### Results of an Experiment Involving the Use of COMPOUND I (imatinib mesylate) in Asbestos-Exposed Mice

Asbestos Exposure: Eight week old C57BL/6 mice are exposed to 15 mg/m³ of chrysotile asbestos in inhalation chambers for a period of 5 hours, for 3 consecutive days, as described previously in detail in our earlier publications (Lasky et al. Am. J. Respir. Crit. Care Med. (1998) 157:1652-7). This asbestos exposure induces fibroblast proliferation and a morphometrically characterized lesion at the alveolar duct bifurcations (Brody et al., Am Rev Respir Dis. (1981) 123:670-9). Sham-exposed (ambient air) animals are used as controls. The groups include: sham-exposed with vehicle; asbestos-exposed with vehicle; and asbestos exposed with COMPOUND I (imatinib mesylate). Five mice from each of these 4 groups are sacrificed 30 days following exposure and used for determining the dimensions of the alveolar duct bifurcations. The 30 day time point is chosen because the fibrotic lesion is predominantly composed of lung myofibroblasts and the connective tissue they maintain 30 days following asbestos exposure.

Administration of COMPOUND I: COMPOUND I (imatinib mesylate) is administered to the mice on the day preceding exposure at the dosage of 100 mg COMPOUND I free base/kg i.p. once per day and on each day thereafter for 14 days. Control mice are given a similar dose of drug-administrating vehicle (DMSO, Tween 80, and saline).

Morphometric Analysis: H & E stained sections from exposed and control animals from the 30 day time point are masked. Bifurcation area is measured using an Olympus microscope with a video camera interfacing with a PC utilizing V150 imaging software (Fermin et al., J Anat. (1995) 186:469-81). The total area and perimeter of each of five first alveolar duct bifurcations is measured as previously described (Perdue et al., J. Histochem. Cytochem. (1994) 42:1061-70). The base of the bifurcation is defined to be delineated by the first lateral alveolar wall to transect the axis of the bifurcation. The resulting measurements from individual animals (5-6 measurements per animal) is averaged to give mean bifurcation dimensions of area and area/perimeter per animal.

Results:

| | First alveolar duct area | |
|---|---|---|
| Group | (square micrometers) | SEM |
| Unexposed | 726 | 140.9 |
| Asbestos alone | 2731 | 218.9 |
| Asbestos/SALT I | 1875 | 308.5 |

Asbestos exposure causes a significant fibrotic scar at first alveolar duct bifurcations (P<0.001). COMPOUND I (imatinib mesylate) administration reduces the size of the lesion by 43% (P=0.02).

These examples illustrate the invention without in any way limiting its scope.

1-4. (canceled)

5. A method of treating a human subject suffering from pulmonary fibrosis disease which comprises administering to said human in need of such treatment a dose, effective against pulmonary fibrosis, of 4-(4-methylpiperazin-1-ylmethyl)-N-[4-methyl-3-(4-pyridin-3-yl)pyrimidin-2-ylamino)phenyl]-benzamide of the formula I or a pharmaceutically acceptable salt thereof.

6. The method according to claim 5 wherein the pulmonary fibrosis disease is induced by asbestos.

7. A method of administering to a human subject suffering from pulmonary fibrosis disease which comprises administering a pharmaceutically effective amount of 4-(4-methylpiperazin-1-ylmethyl)-N-[4-methyl-3-(4-pyridin-3-yl)pyrimidin-2-yl-amino)phenyl]-benzamide of the formula I or a pharmaceutically acceptable salt thereof to said human subject once daily for a period exceeding 3 months.

8. The method according to claim 7 wherein 4-(4-methylpiperazin-1-ylmethyl)-N-[4-methyl-3-(4-pyridin-3-yl)pyrimidin-2-yl-amino)phenyl]-benzamide of the formula I is in the form of a pharmaceutically acceptable acid addition salt thereof.

9. The method according to claim 8 wherein 4-(4-methylpiperazin-1-ylmethyl)-N-[4-methyl-3-(4-pyridin-3-yl)pyrimidin-2-ylamino)phenyl]-benzamide of the formula I is in the monomethane sulfonate salt form thereof.

10. The method according to claim 9 wherein 4-(4-methylpiperazin-1-ylmethyl)-N-[4-methyl-3-(4-pyridin-3-yl)pyrimidin-2-ylamino)phenyl]-benzamide of the formula I is in the beta crystal form of the mesylate salt.

11. A method of administering to a human subject suffering from pulmonary fibrosis disease which comprises administering a daily dose of a monomethanesulfonate salt of 4-(4-methylpiperazin-1-ylmethyl)-N-[4-methyl-3-(4-pyridin-3-yl)pyrimidin-2-ylamino)phenyl]-benzamide of the formula I in the beta crystal form corresponding to 200 to 800 mg of 4-(4-methylpiperazin-1-ylmethyl)-N-[4-methyl-3-(4-pyridin-3-yl)pyrimidin-2-ylamino)phenyl]-benzamide of the formula I free base is administered to an adult human.

12. The method according to claim 5 wherein the pulmonary fibrosis disease is an interstitial lung disease.

13. The method according to claim 5 wherein the pulmonary fibrosis disease is an idiopathic pulmonary fibrosis.
